(19) United States
(12) Patent Application Publication
(10) Pub. No.: US 2004/0126347 A1
(43) Pub. Date: Jul. 1, 2004

(54) Use of fluorinated solvents non-volatile at room temperature in cosmetic or pharmaceutical products

(76) Inventors: Michel Surbled, Saint-Nolff (FR); Bernard Mompon, Vannes (FR)

Correspondence Address: Robert M Angus, Westman Champlin & Kelly, Suite 1600-International Centre, 900 Second Avenue South, Minneapolis, MN, 55402-3319, US

(21) Appl. No.: 10/451,976

(22) PCT Filed: Dec. 27, 2001
(86) PCT No.: PCT/FR01/04218

(30) Foreign Application Priority Data
Dec. 27, 2000 (FR) 00/17146

(51) Int. Cl. A61K 7/06

(52) U.S. Cl. 424/70.1; 510/130

(57) ABSTRACT

The invention concerns the use of fluorinated solvents non-volatile at room temperature in cosmetic or pharmaceutical products. The invention concerns any cosmetic or dermatological product comprising at least a functional and/or active, and/or aromatic and/or dyeing compound, characterised in that said dissolving agent is a hydrofluoroether corresponding to formula: CₙFₘOCₓH_{y}, wherein: the groups CₙHₘ— and/or CₓH_{y}— are linear, branched or cyclized and n ranges between 7 and 12, m ranges between 14 and 25, x ranges between 1 and 8, and y ranges between 3 and 17, said hydrofluoroether being a liquid at room temperature and having a boiling point higher than 100° C.

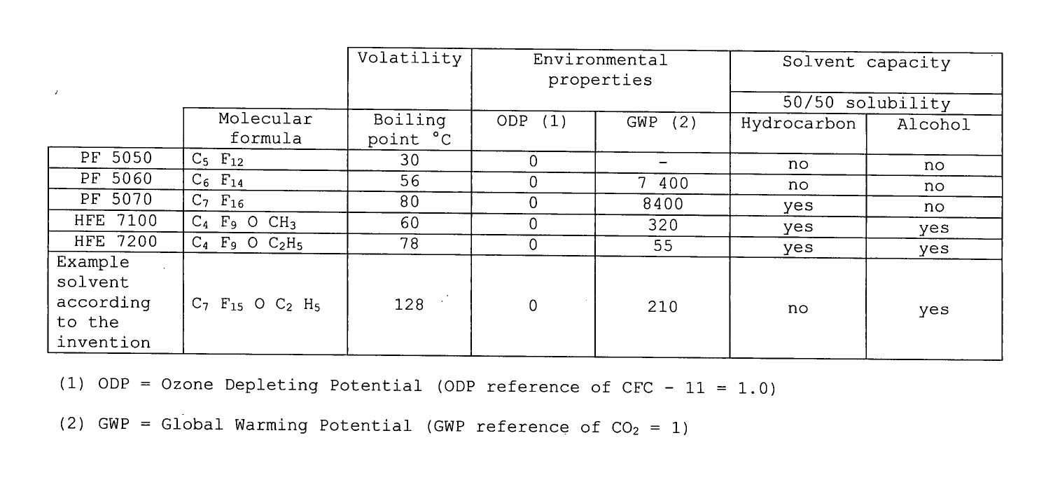

The present invention relates to the domain of cosmetic and pharmaceutical industry.

More precisely, the invention relates to the formulation of cosmetic compositions for, make-up, solar protection, perfume, hair removal cream, deodorants, anti-transpiration or shampoo products, and the formulation of pharmaceutical compositions to be applied to the skin (dermatological compositions).

Traditional cosmetic and dermatological compositions contain ethanol and/or solvents and volatile oils for dissolution of pharmaceutically active and/or odorising and/or colouring constituents.

These solvents have several disadvantages, particularly they have a strong odour and/or are inflammable and/or are polluting to nature (non-zero potential to destroy the ozone layer and contribution to the greenhouse effect). In order to overcome these disadvantages, patent FR2771290 issued by the Applicant proposed to replace conventional solvents by volatile hydrofluoroethers with a boiling temperature of between +15° C. and +100° C. at atmospheric pressure.

Although this type of cosmetic composition is kind towards the ecosystem and also avoids burning sensations related to the use of alcohol, drying of the skin and the appearance of rashes, it is nevertheless true that excessive volatility of products can also be a disadvantage for their use since a non-negligible proportion of consumers do not hermetically and systematically close the receptacles that contain the products after use. Thus, these compositions may be fairly quickly degraded and therefore become unusable or inefficient.

The purpose of this invention is to solve this problem.

Consequently, the invention relates to any cosmetic or dermatological composition comprising at least one functional and/or active and/or aromatic and/or colouring compound and at least one agent for dissolution of the said compound,

characterised in that the said dissolution agent is a hydrofluoroether with the following formula:

CₙFₘOCₓH_{y}

in which the CₙFₘ— and/or CₓH_{y}— groups are linear, ramified or cyclic and in which n is between 7 and 12, m is between 14 and 25, x is between 1 and 8 and y is between 3 and 17,

the said hydrofluorether is a liquid at ambient temperature and has a boiling point of more than 100° C.

Note that the functional and/or active and/or aromatic and/or colouring compounds to be solubilised may be of inorganic or organic, and synthetic or natural origin.

In particular, the functional compounds may be composed of thickening agents, gelling agents, emulsifiers, silicones, and surfactant polymers.

In particular, active compounds may be composed of solar filters, anti-transpiration agents, healing agents, plant extracts (liquids, creams, or powder), anti-radicalar agents, anti-oxidant agents, hydrating agents, anti-bacterial agents, anti-mycosis agents and anti-microbial agents.

In particular, colouring agents may be composed of synthetic or natural pigments used particularly in make up, lipsticks, and foundation applications.

In particular, aromatic compounds may be composed of perfumes and essential oils.

Therefore, the purpose of this invention is the use of at least one fluorinated solvent with a boiling point of more than 100° C. in a cosmetic composition, and that, as can be seen in the table in the single figure, combines the environmental properties of perfluorocarbons (particularly those known to those skilled in the art as PF 5050 to 5070) with low solvent capacities, with the higher solvent capacity of hydrofluorocarbons (particularly the hydrofluorocarbons known to those skilled in the art as denominations HFE 7100 and HFE 7200).

The hydrofluorethers according to the invention have the advantage of not being inoffensive towards the environment and being uninflammable.

Preferably, the said hydrofluorether used to make compositions according to the invention is chosen from the group composed of compounds with the following formulas C₇F₁₅OCH₃, C₇F₁₅OC₂H₅, C₇F₁₅OC₃H₇ and C₇F₁₅OCH₄H₉.

Preferably, the said hydrofluoroether is C₇F₁₅OC₂H₅ with a boiling point of 128° C.

If required, the hydrofluoroethers mentioned may be used in the form of a mix of the linear product and its isomers.

Note that advantageously, the compositions according to the invention will contain between 1 and 95% by weight of the said hydrofluoroether as a percentage of the total weight of the said composition, and that they will preferably contain between 5 and 50% by weight of the said hydrofluoroether as a percentage of the total weight of the said composition.

The composition according to the invention may be aqueous and emulsified, or lipidic and single phase.

According to a first variant, the composition according to the invention consists of a water in oil emulsion made using a natural or synthetic surfactant and is in the form of a gel, a mask or a cream to be applied directly onto the skin for make-up and/or solar protection and/or to avoid sweating and/or to protect the skin and/or clean the skin and/or for skin care.

According to another variant, the said active and or aromatic and/or colouring compound is soluble in the said hydrofluoroether and is in the form of a liquid for odorising and/or making up and/or solar protection of the skin.

According to another variant, the composition according to the invention is in the form of an oil in water dispersion capable of forming composite films on different keratinic substrates.

According to another variant, the composition according to the invention is anhydrous and in particular is in the form of a foundation, a mascara, a lipstick, eye liner, blush or eye shadow.

A non-limitative example of how to make a composition according to the invention is given below.

According to this example, a cream base is produced by mixing a percentage by weight of the following composition:

5% of C₇F₁₅OC₂H₅;

3% of an emulsifying polymer composed of a fatty alcohol and polyethylene glycol ether or a fatty acid ester;

5% of C₁₆ or C₁₈ fatty alcohol;

2% of fatty acid esters such as octyl or isopropyl palmitate or stearate;

2% of glycerol or propylene glycol;

0.5% of a preserving agent: 0,5% such as benzoic acid;

1% of an odorising composition;

Water: q.s. 100%

1. Cosmetic or dermatological composition comprising at least one functional and/or active and/or aromatic and/or colouring compound and at least one agent for dissolution of the said compound,
characterised in that the said dissolution agent is a hydrofluorether with the following formula:
CₙFₘOCₓH_{y}
in which the CₙFₘ— and/or CₓH_{y}— groups are linear, ramified or cyclic and in which n is between 7 and 12, m is between 14 and 25, x is between 1 and 8 and y is between 3 and 17,
the said hydrofluorether is a liquid at ambient temperature and has a boiling point of more than 100° C.

2. Cosmetic or dermatological composition according to claim 1, characterised in that the said hydrofluorether is chosen from the group composed of compounds with the following formulas C₇F₁₅OCH₃, C₇F₁₅OC₂H₅, C₇F₁₅OC₃H₇ and C₇F₁₅OCH₄H₉

3. Cosmetic or dermatological composition according to claim 1, characterised in that the said hydrofluoroether is C₇F₁₅OC₂H₅.

4. Cosmetic or dermatological composition according to any one of claims 1 to 3, characterised in that it contains between 1 and 95% by weight of the said hydrofluoroether as a percentage of the total weight of the said composition.

5. Cosmetic or dermatological composition according to claim 4, characterised in that it contains between 5 and 50% by weight of the said hydrofluoroether as a percentage of the total weight of the said composition.

6. Cosmetic or dermatological composition according to any one of claims 1 to 5, characterised in that it is composed of a water in oil emulsion made using a natural or synthetic surfactant, and is in the form of a gel, a mask or a cream to be applied directly onto the skin for make-up and/or solar protection and/or to avoid sweating and/or to protect the skin and/or clean the skin and/or for skin care.

7. Cosmetic or dermatological composition according to any one of claims 1 to 5, characterised in that the said active and/or aromatic and/or colouring compound is soluble in the said hydrofluoroether and is in the form of a liquid for odorising and/or making up and/or solar protection of the skin.

8. Cosmetic or dermatological composition according to any one of claims 1 to 5, characterised in that it is in the form of an oil in water dispersion capable of forming composite films on different keratinic substrates.

9. Cosmetic or dermatological composition according to any one of claims 1 to 5, characterised in that it is anhydrous and in particular is in the form of a foundation, a mascara, a lipstick, eye liner, blush or eye shadow.
